(19) United States
(12) Patent Application Publication
(10) Pub. No.: US 2022/0347425 A1
(43) Pub. Date: Nov. 3, 2022

(54) VENTILATOR WITH GASPING MODE

(71) Applicant: Keith G. Lurie, Minneapolis, MN (US)

(72) Inventor: Keith G. Lurie, Minneapolis, MN (US)

(21) Appl. No.: 17/735,374

(22) Filed: May 3, 2022

Related U.S. Application Data
(60) Provisional application No. 63/183,494, filed on May 3, 2021.

(51) Int. Cl. A61M 16/20; A61H 31/00; A61M 16/00

(52) U.S. Cl. CPC A61M 16/204; A61M 16/205; A61H 31/006; A61M 16/0003; A61M 2016/0027; A61M 2230/40; A61H 2201/107

(57) ABSTRACT

An automated mechanical ventilator may include a positive pressure source that periodically delivers periodic positive pressure ventilations to a patient when a pressure within the patient's airway is greater than a predetermined threshold. The ventilator may include an inspiratory lumen coupled with the positive pressure source. The ventilator may include an inlet valve interfaced with the inspiratory lumen. The inlet valve may open with each positive pressure ventilation. The ventilator may include an expiratory lumen. The ventilator may include a pressure sensor in fluid communication with the expiratory lumen that senses the pressure within the patient's airway. The ventilator may include an outlet valve interfaced with the expiratory lumen. The ventilator may include a controller that opens the first valve without delivering a positive pressure ventilation when the pressure measured by the pressure sensor is less than the predetermined threshold.

### CROSS-REFERENCE TO RELATED APPLICATIONS

This application claims the benefit of and is a non-provisional of co-pending U.S. Provisional Application Ser. No. 63/183,494 filed on May 3, 2021, which is hereby expressly incorporated by reference in its entirety for all purposes.

### BACKGROUND OF THE INVENTION

Conventional mechanical ventilators deliver positive pressure breaths to patients through an inspiratory lumen and enable gases expelled from the patient to be vented through an expiratory lumen. Such ventilators are often used during resuscitation efforts, such as cardiopulmonary resuscitation (CPR). While effective for performing breathing assistance for unresponsive patients, such ventilator devices do not include modes that enable patients to spontaneously gasp during a resuscitation effort without the immediate and subsequent delivery of a positive pressure breath. Additionally, conventional ventilators do not deliver positive pressure ventilations during CPR at a set rate, inhibit delivery of respiratory gases to the patient during the decompression phase of CPR, and/or synchronize positive pressure ventilation delivery during predominantly the decompression phase of CPR. Therefore, improvements in ventilator devices are desired.

### BRIEF SUMMARY OF THE INVENTION

In one embodiment, an automated mechanical ventilator is provided. The ventilator may include a positive pressure source that periodically delivers periodic positive pressure ventilations to a patient when a pressure within the patient's airway is greater than a predetermined threshold. The positive pressure source may include a pressurized gas, a mechanical piston, a turbine, and/or a mechanically compressible gas chamber (e.g., resuscitator bag). The ventilator may include an inspiratory lumen coupled with the positive pressure source. The ventilator may include an inlet valve interfaced with the inspiratory lumen. The inlet valve may open with each positive pressure ventilation. The ventilator may include an expiratory lumen. The circuit for the ventilator may include and inspiratory and expiratory limb. The ventilator may include one or more pressure sensors in fluid communication with the inspiratory and expiratory lumens that sense the pressure within the patient's airway. The ventilator may include an outlet valve interfaced with the expiratory lumen. The ventilator may include a controller that opens the first valve without delivering a positive pressure ventilation when the pressure measured by the pressure sensor is less than the predetermined threshold.

In another embodiment, a method of providing positive pressure ventilations is provided. The method may include periodically delivering periodic positive pressure ventilations to a patient via an inspiratory lumen of an automated mechanical ventilator. The method may include closing an inlet valve that is coupled with the inspiratory lumen after each positive pressure ventilation. The method may include determining that a pressure within the patient's airway is less than a predetermined threshold. The method may include opening the inlet valve without supplying a positive pressure ventilation based on the determination.

In another embodiment, a method of providing positive pressure ventilations may also include sensing compressions and decompressions, by sensing changes in airway pressure for example, and synchronizing the delivery of the positive pressure breath predominantly during the chest decompression phase of CPR over a period of time between 400-550 ms when CPR is delivered at a rate of 90-110 compressions/minute. The ventilator may include a controller that opens the first valve without delivering a positive pressure ventilation when the pressure measured by the pressure sensor is less than the predetermined threshold. The ventilator may deliver positive pressure ventilations at a constant rate.

### BRIEF DESCRIPTION OF THE DRAWINGS

A further understanding of the nature and advantages of various embodiments may be realized by reference to the following figures. In the appended figures, similar components or features may have the same reference label. Further, various components of the same type may be distinguished by following the reference label by a dash and a second label that distinguishes among the similar components. If only the first reference label is used in the specification, the description is applicable to any one of the similar components having the same first reference label irrespective of the second reference label.

FIG. 1 is a schematic view of an automated mechanical ventilator having a gasping mode according to embodiments of the invention.

FIG. 2 is a schematic view of an automated mechanical ventilator having a gasping mode according to embodiments of the invention.

FIG. 3 is a schematic view of an automated mechanical ventilator having a gasping mode according to embodiments of the invention.

FIG. 4 is a flowchart illustrating a process of providing positive pressure ventilations according to embodiments of the invention.

### DETAILED DESCRIPTION OF THE INVENTION

Embodiments of the present invention are directed to mechanical ventilators and methods of treating patients using such ventilators. In particular, embodiments of the present invention are directed to ventilators and use thereof that enable cardiac arrest patients to spontaneously gasp without the delivery of a positive pressure ventilation during the gasp. While discussed primarily in the context of CPR, it will be appreciated that the ventilators described herein may be usable in numerous other applications in which a patient needs breathing assistance.

During CPR, synchronizing positive breath delivery by an automated mechanical ventilator (AMV) with the compression-decompression cycle provides a number of potential advantages, as such synchronization provides a means to deliver positive pressure ventilation (PPV) primarily during the decompression phase of CPR (thus limiting peak inspiratory pressures) (PIP). The synchronization of PPV using an AMV with the decompression phase of CPR 1) reduces known and common errors with resuscitator bag delivery of PPV such as hyperventilation and excessive tidal volumes, 2) reduces the number of individuals need to care of the patient (1-2 less since PPV is automated), and 3) reduces the pulmonary and hemodynamic consequences associated with delivery a PPV during the compression phase of CPR (which causes excessive PIP and limits the amount of tidal volume delivered). However, no AMV exists that allows the patient to gasp spontaneously and at the same time provide from about 7-16 cm of H2O inspiratory resistance but not have the gasping effort trigger a positive pressure ventilation.

Gasping, which occurs during when the brain is still functional enough to initiate an agonal respiration during CPR, is known to be associated with positive outcomes. In fact, the presence of gasping and a shockable rhythm such as ventricular fibrillation is associated with a 57 times higher likelihood of 1-year survival with favorable brain function than the absence of gasping and a shockable rhythm. (Debaty G, Labarere J, Frascone R J, et al: Long-term prognostic value of gasping during out-of-hospital cardiac arrest. J Am Coll Cardiol 2017; 70:1467-1476) Thus, there is a need for an AMV that can sense a gasp and allow it to occur without the gasp itself triggering a PPV. Currently AMVs can sense a gasping effort, but are programmed to deliver a PPV immediately thereafter in an “assist-control mode.” More specifically, the assist-control mode is typically a mode that is programmed to such that an AMV delivers a set or fixed tidal volume at set intervals of time or when the patient initiates a breath.

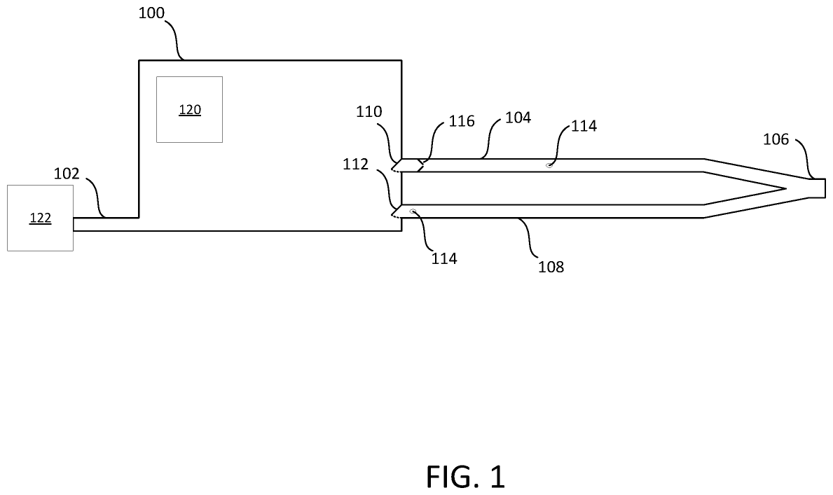

FIG. 1 illustrates a schematic view of one embodiment of an AMV 100 that includes a spontaneous gasping mode. AMV 100 may include a positive pressure source as known in the art, such as a motor or other actuator (not shown), that may generate a positive pressure airflow for delivery to a patient's airways. The positive pressure source may include a pressurized gas, a mechanical piston, a turbine, and/or a mechanically compressible gas chamber (e.g. resuscitator bag). The AMV may be gas or electric powered and will contain a microprocessor and sensors and software to provide, at a minimum, positive pressure ventilation. The AMV 100 may have an inlet 102 that may be coupled with an oxygen source 122, such as an oxygen tank and/or environmental air. AMV 100 may include an outlet that may be coupled with an inspiratory lumen 104, which may deliver oxygen-rich air to a patient. For example, a patient interface (not shown), such a laryngeal mask airway, nasal interface, full face mask, endotracheal tube, a supraglottic airway, and the like, may be coupled with an interface 106 that is coupled with an outlet end of the inspiratory lumen 104. Also coupled with the interface 106 may be an expiratory lumen 108, through which expiratory gases from the patient may be expelled. In some embodiments, an outlet side of the expiratory lumen 108 may be coupled with an expiration port of the AMV 100. In some embodiments the expiratory port may be a non-rebreather valve allowing for inspiration of oxygen-rich respiratory gas, by delivery of a positive pressure breath from the ventilator or by spontaneous inspiratory by the patient, with expiration through a separate one-way valve that is part of the non-rebreather valve.

The AMV 100 may include an inlet valve 110 that is coupled with the inspiratory lumen 104. The inlet valve 110 may be selectively actuated to control the delivery of positive pressure ventilations by the AMV 100. For example, the inlet valve 110 may be closed after delivery of each positive pressure ventilation to seal off the inspiratory lumen 104 until delivery of the next positive pressure ventilation. AMV 100 may include an outlet valve 112 that is coupled with the expiratory lumen 108. Outlet valve 112 may open to expel expiratory gases when pressure within the patient is greater than about 0 atmospheres. Each valve may be coupled with a respective actuator (not shown) that may be used to open and close the respective valve. Valves 110, 112 may take any form, including duck bill valves, diaphragms, fish mouth valves, ball valves, check valves, flap gate valves, butterfly valves, and the like. A pressure sensor 114 may be in fluid connection with the patient, such as disposed within the expiratory lumen 108, and may be used to sense changes in pressure within the patient's lungs.

AMV 100 may include a controller that controls operation of the actuators and valves 110, 112 to control delivery of positive pressure ventilations to a patient. In a normal mode, the controller may synchronize positive pressure delivery with a decompression phase of CPR. For example, the controller may sense a peak compression of the patient's chest using a measurement from the pressure sensor 114 (or from a signal from an automated chest compression and/or decompression device). The sensed peak pressure may cause the controller to trigger the inlet valve 110 to open (while outlet valve 112 is closed) and may cause a positive pressure ventilation to be delivered to the patient. Positive pressure ventilations may be delivered to the patient every 8-12 compressions (with a compression rate of between about 90 and 120 compressions per minute) at peak compression. A duration of each individual positive pressure ventilation may be between about 400 and 1200 ms, commonly between 450 and 900 ms, and more commonly about 500 ms. After each positive pressure breath, the inlet valve 110 may be closed. Closing the inlet valve 110 after each positive pressure ventilation may inhibit delivery of respiratory gases to the patient during at least a portion of the decompression phase of CPR, which may enhance the negative pressure within the thorax. This enhanced vacuum leads to greater the refilling of the heart and the egress of venous blood from the brain and may increase significantly 1-year survival after cardiac arrest when AMV 100 is used concurrently with active compression-decompression CPR.

The controller may automatically switch operation of the AMV 100 to a gasping mode upon sensing a spontaneous gasp from a patient. In some instances the controller may be preprogrammed when in the CPR mode to be in the gasping mode. When in the gasping mode, there may be two special features that triggered, either separately or together. First, if a gasp is detected, the gasp is not automatically followed by an assisted ventilation, which is often how ventilators are programmed to function. That is, if an inspiratory effort is detected, commonly the ventilator is programmed to deliver a positive pressure ventilation to ‘assist’ the effort by the patient to inspire. In the present invention, no ‘assisted breath’ would be delivered upon detection of a gasping effort, when airway pressures are sensed to be below a programmed threshold value of −1 to −16 cm H2O. Rather, the ventilator would deliver a synchronized breath primarily during the decompression phase of the CPR cycle at a regular preset interval, typically 8-12 times per minute. Second, when an inspiratory effort is detected the inlet valve 100 would open fully or partially to allow for inflow of oxygen-rich respiratory gases into the patient with up to 16 cm H2O at a flow rate of between 30-50 L/min. In this way, the patient's inspiratory effort lowers intrathoracic pressure, which draws both oxygen into the lungs through inlet valve 110, and venous blood back to the heart from the rest of the body including the brain. Thus, the patient benefits from the spontaneous gasping effort clinically without either getter an ‘assisted’ breath with each gasp or alternatively not being able to inhale, in the case when inlet valve 110 remains closed upon detecting a gasp, except when the ventilator delivers a positive pressure breath at preset regular intervals. In other words, in the gasping mode, the ventilator provides adequate ventilation and allows the patient to gasp in a manner that allows the patient to benefit from this natural reflex to enhance circulation and oxygen delivery. [ref Debaty G, Labarere J, Frascone R J, et al: Long-term prognostic value of gasping during out-of-hospital cardiac arrest. J Am Coll Cardiol 2017; 70:1467-1476].

For example, when in the gasping mode or CPR mode with gasping detect, upon detecting that the pressure within the patient is less than a predetermined threshold (oftentimes between −7 to −16 cm H2O and commonly about −12 cm H2O), the controller may determine that the patient has spontaneous gasped, as pressures more negative than that predetermined threshold do not occur with CPR alone, but only with spontaneous gasping effort. Upon such a determination, the controller may cause an actuator to open valve 110 (fully or partially), without providing positive pressure airflow through the inspiratory lumen 104. Should this occur, valve 110 may open partially thereby providing resistance of up 0 to −16 cm H2O, preferably −7 to −16 cm H2O, at a flow rate of between 20-50 L/min. In other words, once the AMV 100 senses a gasp, the inlet valve 110 may remain closed until a predetermined threshold of −7 to −16 cm H2O resistance and then it opens fully or partially. In this manner no or some low level of resistance may still intentionally be provided during the gasping effort, but a positive pressure ventilation may not be delivered until the next positive pressure ventilation is preprogrammed to be delivered based upon a regular breath delivery timing interval. For example, in some embodiments, if a scheduled positive pressure ventilation coincides with the gasp, that positive pressure ventilation may be skipped such that no positive pressure ventilation is delivered during the gasping effort, and the subsequently scheduled positive pressure ventilation may be delivered at its original interval. In other embodiments, the positive pressure ventilations may be ceased for a predetermined period of time (e.g., 5 seconds, 10 seconds, etc.) after a gasping effort is detected. In yet other embodiments, the operation of the positive pressure source 120 may not be impacted by the detection of a gasping effort.

Importantly, in the gasping mode, the sensed gasp or patient effort to inspire does not trigger a positive pressure ventilation as long as the sensor 114, or some other means (such as based on a signal from the chest compression and/or decompression device), detects the presence of ongoing CPR. In other words, with chest compression the airway pressure rises and falls at CPR rates of 80-120 times per minute. This enables the patient to gasp, or in fact, breathe through AMV 100, which may enhance circulation during CPR. When in the gasping mode, the AMV 100 will not deliver a positive pressure ventilation with each gasp as long as CPR is ongoing. When CPR is no longer detected (using sensor 114 and/or signals from a chest compression device and/or user input), the AMV 100 may automatically switch to an assist-control mode in which a positive pressure breath is delivered when spontaneous inspiratory efforts are detected.

In some embodiments the physiological benefits of the CPR mode with gasping detect may be accomplished by a plurality of inlet valves in-line within the inspiratory circuit. For example, as shown in FIG. 1 inlet valve 110 may open fully upon detection of a gasp when pressures sensed directly or indirectly within the patient fall below a preprogrammed detection threshold value of −12 to −16 cm H2O. When fully open valve 110 may provide no inspiratory resistance by itself. However, a one-way resistance valve 116, proximal to inlet valve 110 (upstream and/or downstream of the inlet valve 110) may have a fixed resistance of, for example, from −10 to −16 cm H2O at a flow over a range of flow values from about 20-50 L/min. Thus, with gasping the patient is able to inspire against a fixed but relatively low level of resistance, thus deriving clinical benefit.

In another embodiment, when the AMV is in the gasping mode or CPR mode with gasping detect, the AMV may be programmed to alter the positive pressure ventilation rate and/or tidal volume depending upon the frequency of gasping. For example, if the gasping rate is detected to be 6 times per minute, then the positive pressure ventilation rate may be reduced from 10 breaths per minute, for example, to 5 breaths per minute. Additional physiological data from non-invasive sensors, for example oxygen saturation, cerebral oximetry, and/or end tidal CO2, may be used together with the frequency of gasping, to further adjust the breath delivery rate based upon an algorithm preprogrammed into the AMV.

Without the gasping mode in an AMV, inspiration associated with spontaneous gasping may be blocked and the gasping effort may result in harmful very negative intrathoracic pressures, which are known to cause negative pressure pulmonary edema. Thus, embodiments of the present invention allow the patient to benefit from spontaneous gasping to enhance oxygen delivery and circulation, a direct physiological benefit of a gasp, and prevent generation of excessive negative intrathoracic pressures which can be harmful when inspiring against a closed airway. When CPR is no longer being performed the AMV 100 switches back to a more typical ‘breathing-assist’ mode. This switch back can be performed manually by the rescuer or preferably in an automated fashion when the AMV 100 no longer detects CPR is ongoing. For example, the AMV no longer senses changes in airway pressure which occur during CPR or is coupled to a sensor that detects chest compression are occurring.

In some embodiments, AMV 100 may include flow sensors that are interfaced with the inspiratory limb 106. The flow sensors may be utilized to determine a volume of air drawn into the lungs by a gasping effort. In some embodiments, the flow volume may be used to determine whether a gasping efforts may occurred and/or may be used to adjust the operation of the AMV 100. For example, if a flow volume is greater than a preset threshold, such as about 500 mL, a gasp may be detected, which may alter the operation of the positive pressure source 120 as described above.

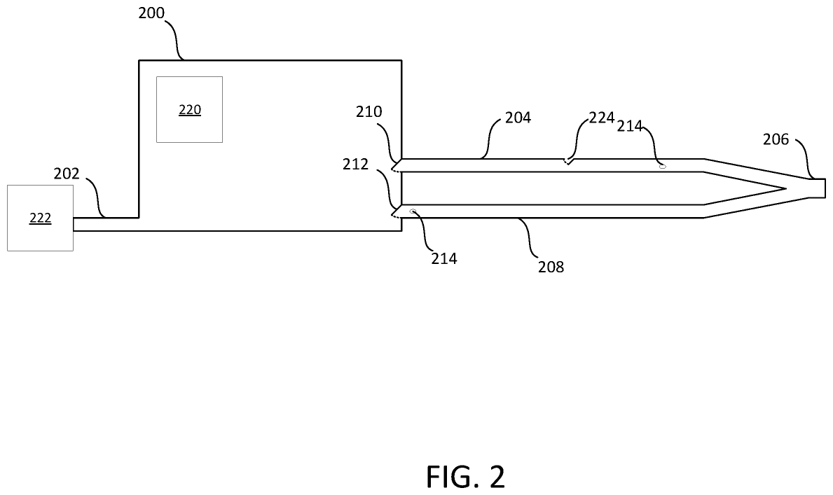

FIG. 2 illustrates a schematic view of one embodiment of an AMV 200 that includes a spontaneous gasping mode and utilizes a passive valve system. AMV 200 may include a positive pressure source as known in the art, such as a motor or other actuator (not shown), that may generate a positive pressure airflow for delivery to a patient's airways. AMV 200 may have an inlet 202 that may be coupled with an oxygen source 222. AMV 200 may include an outlet that may be coupled with an inspiratory lumen 204, which may deliver oxygen-rich air to a patient. Also coupled with the interface 206 may be an expiratory lumen 208, through which expiratory gases from the patient may be expelled. In some embodiments, an outlet side of the expiratory lumen 208 may be coupled with an expiration port of the AMV 200. In some embodiments the expiratory port may be a non-rebreather valve allowing for inspiration of oxygen-rich respiratory gas, by delivery of a positive pressure breath from the ventilator or by spontaneous inspiratory by the patient, with expiration through a separate one-way valve that is part of the non-rebreather valve.

The AMV 200 may include an inlet valve 210 that is coupled with the inspiratory lumen 204. The inlet valve 210 may be a one-way valve that opens when the positive pressure source 220 delivers a positive pressure ventilation to the patient and may be biased to close after delivery of each positive pressure ventilation to seal off the inspiratory lumen 204 until delivery of the next positive pressure ventilation. AMV 200 may include an outlet valve 212 that is coupled with the expiratory lumen 208. Outlet valve 212 may be a one-way valve that opens to expel expiratory gases when pressure within the patient is greater than a predetermined cracking pressure, which may be about 0 atmospheres in some embodiments. Valves 210, 212 may take any form, including duck bill valves, diaphragms, fish mouth valves, ball valves, check valves, flap gate valves, butterfly valves, and the like.

The AMV may include a one-way valve 224 that is coupled with the inspiratory lumen 206. The one-way valve 224 may be designed to open when a spontaneous gasp occurs. For example, the inlet valve 224 may provide a predetermined amount of resistance, such as between about −7 to −16 cm H2O. When a patient gasps and generates a negative intrathoracic pressure that exceeds the cracking pressure of the one-way valve 224, the one-way valve 224 opens fully or partially to allow for inflow of oxygen-rich respiratory gases (such as from the environment) into the patient with up to 16 cm H2O at a flow rate of between 30-50 L/min. In this way, the patient's inspiratory effort lowers intrathoracic pressure, which draws both oxygen into the lungs through one-way valve 224, and venous blood back to the heart from the rest of the body including the brain. Thus, the patient benefits from the spontaneous gasping effort clinically without either getter an ‘assisted’ breath with each gasp or alternatively not being able to inhale.

In some embodiments, AMV 200 may include one or more pressure sensors 214 that may be in fluid connection with the patient. For example, a pressure sensor 214 may be disposed within the inspiratory lumen 206 and/or the expiratory lumen 208, and may be used to sense changes in pressure within the patient's lungs. AMV 200 may include a controller that controls operation of the positive pressure source 220 to control delivery of positive pressure ventilations to a patient. In a normal mode, the controller may synchronize positive pressure delivery with a decompression phase of CPR. For example, the controller may sense a peak compression of the patient's chest using a measurement from the pressure sensor 214 (or from a signal from an automated chest compression and/or decompression device). The sensed peak pressure may cause the controller to cause a positive pressure ventilation to be delivered to the patient in a manner similar to that described in relation to AMV 100. After each positive pressure breath, the inlet valve 210 may close. Closing the inlet valve 210 after each positive pressure ventilation may inhibit delivery of respiratory gases to the patient during at least a portion of the decompression phase of CPR, which may enhance the negative pressure within the thorax. The controller may automatically switch operation of the AMV 200 to a gasping mode upon sensing a spontaneous gasp from a patient in a manner similar to as described in relation to AMV 100. For example, if a gasp is detected (e.g., airway pressures are sensed to be below a programmed threshold value of −1 to −16 cm H2O), the gasp is not automatically followed by an assisted ventilation. Rather, the ventilator would deliver a synchronized breath primarily during the decompression phase of the CPR cycle at a regular preset interval, typically 8-12 times per minute.

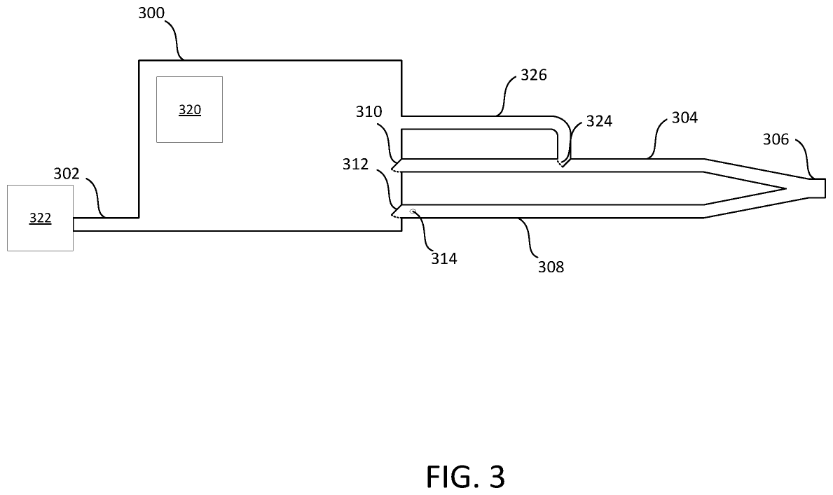

FIG. 3 illustrates a schematic view of one embodiment of an AMV 300. AMV 300 may include a positive pressure source as known in the art, such as a motor or other actuator (not shown), that may generate a positive pressure airflow for delivery to a patient's airways. AMV 300 may have an inlet 302 that may be coupled with an oxygen source 322. AMV 300 may include an outlet that may be coupled with an inspiratory lumen 304, which may deliver oxygen-rich air to a patient. A gasping lumen 326 may be fluidly coupled with the inspiratory lumen 304, with flow between the two lumens being controlled by a one-way valve 324. Also coupled with the interface 306 may be an expiratory lumen 308, through which expiratory gases from the patient may be expelled. In some embodiments, an outlet side of the expiratory lumen 308 may be coupled with an expiration port of the AMV 300. In some embodiments the expiratory port may be a non-rebreather valve allowing for inspiration of oxygen-rich respiratory gas, by delivery of a positive pressure breath from the ventilator or by spontaneous inspiratory by the patient, with expiration through a separate one-way valve that is part of the non-rebreather valve.

The AMV 300 may include an inlet valve 310 that is coupled with the inspiratory lumen 304. The inlet valve 310 may be a one-way valve that opens when the positive pressure source 320 delivers a positive pressure ventilation to the patient and may be biased to close after delivery of each positive pressure ventilation to seal off the inspiratory lumen 304 until delivery of the next positive pressure ventilation. AMV 300 may include an outlet valve 312 that is coupled with the expiratory lumen 208. Outlet valve 312 may be a one-way valve that opens to expel expiratory gases when pressure within the patient is greater than a predetermined cracking pressure, which may be about 0 atmospheres in some embodiments. Valves 310, 312 may take any form, including duck bill valves, diaphragms, fish mouth valves, ball valves, check valves, flap gate valves, butterfly valves, and the like.

The one-way valve 324 may be designed to open when a spontaneous gasp occurs. For example, the inlet valve 324 may provide a predetermined amount of resistance, such as between about −7 to −16 cm H2O. When a patient gasps and generates a negative intrathoracic pressure that exceeds the cracking pressure of the one-way valve 324, the one-way valve 324 opens fully or partially to allow for inflow of oxygen-rich respiratory gases (such as from the environment) via the gasping lumen 326 into the patient with up to 16 cm H2O at a flow rate of between 30-50 L/min. In this way, the patient's inspiratory effort lowers intrathoracic pressure, which draws both oxygen into the lungs through one-way valve 324, and venous blood back to the heart from the rest of the body including the brain. Thus, the patient benefits from the spontaneous gasping effort clinically without either getter an ‘assisted’ breath with each gasp or alternatively not being able to inhale.

In some embodiments, AMV 300 may include one or more pressure sensors 314 that may be in fluid connection with the patient. For example, a pressure sensor 314 may be disposed within the inspiratory lumen 306 and/or the expiratory lumen 308, and may be used to sense changes in pressure within the patient's lungs. AMV 300 may include a controller that controls operation of the positive pressure source 320 to control delivery of positive pressure ventilations to a patient. In some embodiments, the controller may synchronize positive pressure delivery with a decompression phase of CPR. For example, the controller may sense a peak compression of the patient's chest using a measurement from the pressure sensor 314 (or from a signal from an automated chest compression and/or decompression device). The sensed peak pressure may cause the controller to cause a positive pressure ventilation to be delivered to the patient in a manner similar to that described in relation to AMV 100. After each positive pressure breath, the inlet valve 310 may close. Closing the inlet valve 310 after each positive pressure ventilation may inhibit delivery of respiratory gases to the patient during at least a portion of the decompression phase of CPR, which may enhance the negative pressure within the thorax. In other embodiments, the controller may be programmed to deliver breathes at a controlled rate at predetermined intervals without any sensor information.

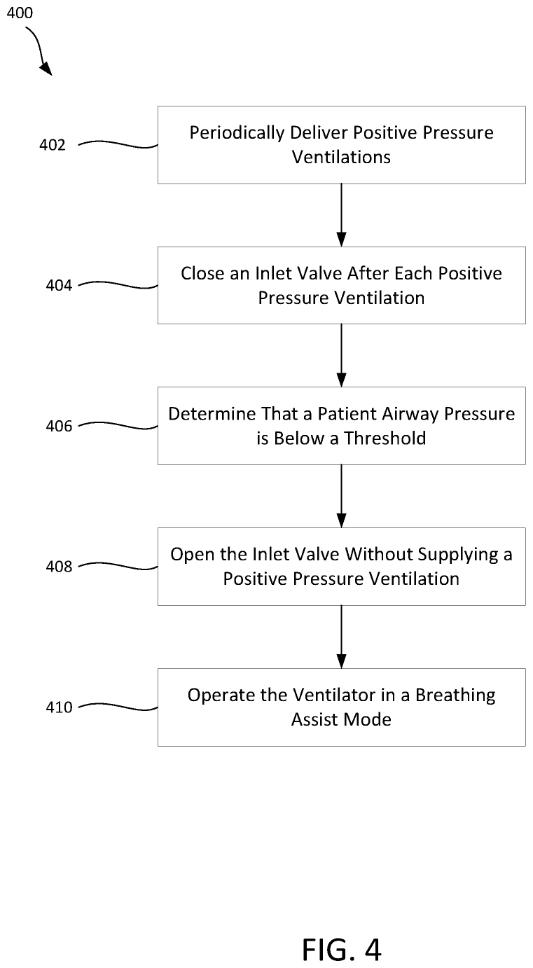

FIG. 4 is a flowchart illustrating a process 400 for delivering positive pressure ventilations. Process 400 may be performed using an automated mechanical ventilator, such as AMV 100, 200, or 300 described above. Process 400 be initiated when an AMV either senses CPR is ongoing by detecting changes in airway pressures generated by chest compressions and/or decompressions or receives an external signal that chest compressions are ongoing, from, for example, a chest compressor or chest motion detector or the like. Process 400 may begin at operation 402 by periodically delivering periodic positive pressure ventilations to a patient via an inspiratory lumen of an AMV. The positive pressure ventilations may be delivered between every 8-12 chest compressions (which may be performed at a rate of between about 90 and 120 compressions per minute) and each positive pressure ventilation may have a duration of between about 400 ms and 1200 ms. Delivery of each positive pressure ventilation may be synchronized with a decompression phase of CPR. For example, the automated mechanical ventilator may synchronize the delivery of positive pressure ventilations based on a signal received from a chest compression device that indicates and end of a compression phase/beginning of a decompression phase and/or by detecting a peak pressure within the patient's airway using one or more pressure sensors.

After each positive pressure, an inlet valve that is coupled with the inspiratory lumen may be closed at operation 404. An outlet valve of the automated mechanical ventilator may be opened when the pressure within the patient's airway is greater than about 0 atmospheres. At operation 406, a determination may be made that a pressure within the patient's airway is less than a predetermined threshold, which may indicate that the patient has spontaneously gasped. In some embodiments, the predetermined threshold may be less than about −7 cm H2O and in other embodiments up to −16 cm H2O. Based on this determination, the inlet valve may be opened (partially or fully) without supplying a positive pressure ventilations based on the determination at operation 408. This may facilitate the patient's gasping attempt and may increase circulation. Once the determination is made that the pressure within the patient's airway is less than the predetermined threshold, the process 400 may include halting delivery of positive pressure ventilations until CPR is no longer being performed. Determining that CPR is no longer being performed may be based on a signal from a chest compression device and/or based on the pressure within the patient's airway or by observations by a rescuer at the scene. Upon determining that CPR is no longer being performed, process 400 may include automatically switching to an assist-control mode in which a positive pressure breath is delivered each time a spontaneous inspiratory breathing effort is detected at operation 410.

FIG. 5 illustrates a block diagram of an example computer system 500 usable for performing image analysis, normalization, and display. The computing device 500 can be or include, for example, a laptop computer, desktop computer, tablet, e-reader, smart phone or mobile device, smart watch, personal data assistant (PDA), or other electronic device.

The computing device 500 can include a processor 540 interfaced with other hardware via a bus 505. A memory 510, which can include any suitable tangible (and non-transitory) computer readable medium, such as RAM, ROM, EEPROM, or the like, can embody program components (e.g., instructions 515) that configure operation of the computing device 500. In some examples, the computing device 500 can include input/output (“I/O”) interface components 525 (e.g., for interfacing with a display 545, keyboard, or mouse) and additional storage 530.

The computing device 500 can include network components 520. Network components 520 can represent one or more of any components that facilitate a network connection. In some examples, the network components 520 can facilitate a wireless connection and include wireless interfaces such as IEEE 802.11, Bluetooth, or radio interfaces for accessing cellular telephone networks (e.g., a transceiver/antenna for accessing CDMA, GSM, UMTS, or other mobile communications network). In other examples, the network components 520 can be wired and can include interfaces such as Ethernet, USB, or IEEE 1394. These communication means may be used for data transfer and real-time control of the AMV system.

Although FIG. 5 depicts a single computing device 500 with a single processor 540, the system can include any number of computing devices 500 and any number of processors 540. For example, multiple computing devices 500 or multiple processors 540 can be distributed over a wired or wireless network (e.g., a Wide Area Network, Local Area Network, or the Internet). The multiple computing devices 500 or multiple processors 540 can perform any of the steps of the present disclosure individually or in coordination with one another.

Each of the instructions, calculations, or operations described herein may be performed using a computer or other processor having hardware, software, and/or firmware. The various method steps may be performed by modules, and the modules may comprise any of a wide variety of digital and/or analog data processing hardware and/or software arranged to perform the method steps described herein. The modules optionally comprising data processing hardware adapted to perform one or more of these steps by having appropriate machine programming code associated therewith, the modules for two or more steps (or portions of two or more steps) being integrated into a single processor board or separated into different processor boards in any of a wide variety of integrated and/or distributed processing architectures. These methods and systems will often employ a tangible media embodying machine-readable code with instructions for performing the method steps described above. Suitable tangible media may comprise a memory (including a volatile memory and/or a non-volatile memory), a storage media (such as a magnetic recording on a floppy disk, a hard disk, a tape, or the like; on an optical memory such as a CD, a CD-R/W, a CD-ROM, a DVD, or the like; or any other digital or analog storage media), or the like. The instructions or operations may be stored in the non-transitory memory or memory device and executed by the processor, which causes the processor to perform the instructions or operations described.

Specific details are given in the description to provide a thorough understanding of example configurations (including implementations). However, configurations may be practiced without these specific details. For example, well-known processes, structures, and techniques have been shown without unnecessary detail in order to avoid obscuring the configurations. This description provides example configurations only, and does not limit the scope, applicability, or configurations of the claims. Rather, the preceding description of the configurations will provide those skilled in the art with an enabling description for implementing described techniques. Various changes may be made in the function and arrangement of elements without departing from the spirit or scope of the disclosure. Additionally, features described in relation to one embodiment may be incorporated into other embodiments while staying within the scope of the disclosure.

Also, configurations may be described as a process that is depicted as a flow diagram or block diagram. Although each may describe the operations as a sequential process, many of the operations may be performed in parallel or concurrently. In addition, the order of the operations may be rearranged. A process may have additional steps not included in the figure.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1. An automated mechanical ventilator, comprising:
a positive pressure source that periodically delivers periodic positive pressure ventilations to a patient when a pressure within the patient's airway is greater than a predetermined threshold;
an inspiratory lumen coupled with the positive pressure source;
an inlet valve interfaced with the inspiratory lumen, the inlet valve opening with each positive pressure ventilation;
an expiratory lumen;
a pressure sensor in fluid communication with the expiratory lumen that senses the pressure within the patient's airway;
an outlet valve interfaced with the expiratory lumen; and
a controller that opens the inlet valve without delivering a positive pressure ventilation when the pressure measured by the pressure sensor is less than the predetermined threshold.

2. The automated mechanical ventilator of claim 1, wherein:
the controller is communicatively coupled with a chest compression device; and
the controller synchronizes delivery of positive pressure ventilations predominantly with a decompression phase of CPR based on one or both of a signal from the chest compression device and a signal from a chest displacement sensor.

3. The automated mechanical ventilator of claim 1, wherein:
the controller synchronizes delivery of positive pressure ventilations with a peak pressure sensed by the pressure sensor.

4. The automated mechanical ventilator of claim 1, wherein:
the predetermined threshold is between about −7 cm H2O and −16 cm H2O.

5. The automated mechanical ventilator of claim 1, wherein:
the inlet valve closes after delivery of each periodic pressure ventilation.

6. The automated mechanical ventilator of claim 1, wherein:
positive pressure ventilations are delivered between every 8-12 chest compressions; and
each positive pressure ventilation has a duration of between about 400 ms and 1200 ms.

7. The automated mechanical ventilator of claim 1, wherein:
the controller determines that CPR is no longer being performed based on one or both of a signal from a chest compression device and the pressure sensed by the pressure sensor.

8. The automated mechanical ventilator of claim 1, wherein:
upon determining that CPR is no longer being performed, the controller automatically switches to an assist-control mode in which a positive pressure breath is delivered each time a spontaneous inspiratory breathing effort is detected.

9. The automated mechanical ventilator of claim 1, wherein:
the inlet valve comprises a plurality of valves, with a first valve that opens fully and a second valve that provides a level of resistance of between 7 and 16 cm of H2O resistance when the patient inspires.

10. A method of providing positive pressure ventilations, comprising:
determining that CPR is being performed on a patient;
periodically delivering periodic positive pressure ventilations to the patient via an inspiratory lumen of an automated mechanical ventilator;
closing an inlet valve that is coupled with the inspiratory lumen after each positive pressure ventilation;
determining that a pressure within the patient's airway is less than a predetermined threshold; and
at least partially opening the inlet valve without supplying a positive pressure ventilation based on the determination.

11. The method of providing positive pressure ventilations of claim 10, wherein:
delivery of the positive pressure ventilations is synchronized with a portion of the decompression and compression phases of CPR.

12. The method of providing positive pressure ventilations of claim 10, further comprising:
upon determining that the pressure within the patient's airway is less than the predetermined threshold, halting delivery of positive pressure ventilations until CPR is no longer being performed.

13. The method of providing positive pressure ventilations of claim 10, further comprising:
determining that CPR is no longer being performed based on a signal from a chest compression device.

14. The method of providing positive pressure ventilations of claim 10, further comprising:
determining that CPR is no longer being performed based on the pressure within the patient's airway.

15. The method of providing positive pressure ventilations of claim 10, further comprising:
determining that CPR is no longer being performed; and
upon determining that CPR is no longer being performed, automatically switching to an assist-control mode in which a positive pressure breath is delivered each time a spontaneous inspiratory breathing effort is detected.

16. The method of providing positive pressure ventilations of claim 10, wherein:
determining that a pressure within the patient's airway is less than a predetermined threshold comprises sensing a gasping effort by the patient.

17. An automated mechanical ventilator, comprising:
a positive pressure source that periodically delivers periodic positive pressure ventilations to a patient;
an inspiratory lumen coupled with the positive pressure source;
an inlet valve interfaced with the inspiratory lumen, the inlet valve opening with each positive pressure ventilation;
a one-way valve interfaced with the inspiratory lumen, the one-way valve having a predetermined cracking pressure;
an expiratory lumen; and
an outlet valve interfaced with the expiratory lumen.

18. The automated mechanical ventilator of claim 17, further comprising:
a pressure sensor interfaced with one or both of the inspiratory lumen and the expiratory lumen, wherein the positive pressure source is configured to deliver the periodic positive pressure ventilations when the pressure sensor detects that a pressure within the patient's airway is greater than a predetermined threshold.

19. The automated mechanical ventilator of claim 18, wherein:
the predetermined threshold is between about −7 cm H2O and −16 cm H2O.

20. The automated mechanical ventilator of claim 17, wherein:
the one-way valve is passively actuated when a pressure in the patient's chest is more negative than the cracking pressure.
